(19) United States
(12) Patent Application Publication
(10) Pub. No.: US 2025/0041196 A1
(43) Pub. Date: Feb. 6, 2025

(54) Oral Care Compositions with Hyaluronic Acid

(71) Applicant: Colgate-Palmolive Company, New York, NY (US)

(72) Inventors: Yuzhi DENG, Guangzhou (CN); Dandan CHEN, Bridgewater, NJ (US); Diana Liceth HENAO ADLEVANKIN, Martinsville, NJ (US); Joseph Allan McKinnon STEELE, Plainfield, NJ (US); Julia Maighdlin DUGDALE, East Windsor, NJ (US); Paul THOMSON, Piscataway, NJ (US); Payal ARORA, Lebanon, NJ (US); Manisha JHA, Houston, TX (US); Lisa Marie MANUS, Lawrenceville, NJ (US); Ying YANG, Monmouth Junction, NJ (US)

(73) Assignee: Colgate-Palmolive Company, New York, NY (US)

(21) Appl. No.: 18/717,936

(22) PCT Filed: Dec. 7, 2022
(86) PCT No.: PCT/CN2022/137219

§ 371 Date: Jun. 7, 2024

(30) Foreign Application Priority Data
Dec. 8, 2021 (CN) 202111491775.X

(51) Int. Cl. A61K 8/73; A61K 8/34; A61K 8/46; A61K 8/49; A61K 8/67; A61Q 11/00

(52) U.S. Cl. CPC A61K 8/735; A61K 8/34; A61K 8/466; A61K 8/4926; A61K 8/676; A61Q 11/00; A61K 2800/48

(57) ABSTRACT

Oral care compositions and methods for treating, preventing, or inhibiting dry mouth and/or inflammation of the oral cavity or a surface thereof are disclosed. Oral care compositions for improving oral care by treating, preventing, or otherwise inhibiting discomfort, irritation, or gum pain of the oral cavity are also disclosed. The oral care composition may include an orally acceptable vehicle, hyaluronic acid, and one or more benefit agents. The method for treating, preventing, or inhibiting dry mouth and/or inflammation of the oral cavity or a surface thereof may include contacting the oral cavity or the surface thereof with the oral care compositions.

### CROSS-REFERENCE TO RELATED APPLICATIONS

This application claims priority to Chinese Patent Application No. 202111491775.X, filed on Dec. 8, 2021, the contents of which are incorporated herein by reference to the extent consistent with the present disclosure.

### BACKGROUND

Dry mouth, often referred to as xerostomia, is a condition in which the salivary glands do not make enough saliva to maintain moisture in the oral cavity. Degrees of dry mouth may range from a nuisance causing minor discomfort, oral malodor, and/or oral stress to something that may greatly impact overall health and/or the health of the oral cavity (e.g., teeth and gums). Similar to the effects of dry mouth, the causes of dry mouth vary greatly. For example, dry mouth may often be the result of medical treatments and/or medications. Dry mouth may also be the result of aging and/or underlying conditions, such as diabetes, cancer, or the like.

Oral inflammation, such as from periodontitis or gum disease and gingivitis, is an inflammatory condition affecting the tissues of the oral cavity. Oral inflammation does not only result in localized issues such as damage to the gums that support your teeth, irritation, and redness, it also can affect other parts of your body. For example, periodontitis has been linked with respiratory disease, rheumatoid arthritis, coronary artery disease, as well as problem with controlling blood sugar in diabetes.

In view of the foregoing, it is clear that the oral cavity may suffer from a variety of conditions. There is, however, a lack of therapeutic and/or non-therapeutic treatments for addressing the myriad of conditions of the oral cavity.

What is needed, then, are improved oral care compositions and methods for the therapeutic and/or non-therapeutic treatment of these oral conditions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

The foregoing and/or other aspects and utilities embodied in the present disclosure may be achieved by providing an oral care composition including an orally acceptable vehicle, hyaluronic acid, and one or more benefit agents.

In at least one implementation, the hyaluronic acid may include one or more of hyaluronic: acid, a salt of hyaluronic acid, a derivative of hyaluronic acid, or combinations thereof, preferably, the hyaluronic acid may include a salt of hyaluronic acid, more preferably, the salt of hyaluronic acid may include sodium hyaluronate.

In at least one implementation, the hyaluronic acid may be present in an amount effective to treat, prevent, or inhibit inflammation of the oral cavity, preferably, the hyaluronic acid may be present in an amount of from about 0.05 wt % to about 0.5%, preferably, about 0.05 wt % to about 0.2 wt %, more preferably about 0.1 wt %, based on the total weight of the composition.

In at least one implementation, the hyaluronic acid may include one or more of low-molecular weight hyaluronic acid, middle molecular weight hyaluronic acid, high-molecular weight hyaluronic acid, or combinations thereof, preferably, the hyaluronic acid may include at least low-molecular weight hyaluronic acid.

In at least one implementation, the orally acceptable vehicle may include one or more of a thickening agent, a humectant, a solvent, a pH modifying agent, a flavorant, or combinations thereof, preferably, the orally acceptable vehicle may include a combination of the thickening agent, the humectant, the solvent, the pH modifying agent, and the flavorant.

In at least one implementation, the thickening agent may include an anionic polymeric thickener, preferably, an acrylate copolymer, an acrylate-alkyl acrylate copolymer, or combinations thereof, more preferably the thickening agent may include an acrylates/C10-30 alkyl acrylate crosspolymer.

In at least one implementation, the thickening agent may be present in an amount of from about 0.01 wt % to about 30 wt %, preferably, about 0.05 wt % to about 15 wt %, more preferably about 0.1 wt % to about 5 wt %, even more preferably about 0.1 wt % to about 2 wt %, or about 1 wt %, based on the total weight of the composition.

In at least one implementation, the humectant may include one or more polyols, preferably, the humectant may include one or more of xylitol, glycerin, or combinations thereof, more preferably the humectant may include a combination of xylitol and glycerin.

In at least one implementation, the humectant may be present in an amount of from about 25 weight % to about 55 weight %, preferably about 30 weight % to about 50 weight %, more preferably about 35 weight % to about 45 weight %, or about 40 weight %, based on the total weight of the composition.

In at least one implementation, the solvent may include water, preferably, a weight ratio of water to glycerin is less than or equal to 1.6:1, less than or equal to 1.5:1, less than or equal to 1.4:1, less than or equal to 1.3:1, less than or equal to 1.2:1, or less than or equal to 1.1:1.

In at least one implementation, the benefit agents may include one or more of a zinc source, vitamin C, tetrahydrodiferuloylmethane, clove oil, hemp seed oil, one or more amino acids, or combinations thereof.

In at least one implementation, the benefit agent may include the zinc source, preferably, the zinc source may include zinc phosphate hydrate, more preferably, the zinc source may be present in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition.

In at least one implementation, the benefit agent may include vitamin C, wherein vitamin C comprises one or more of ascorbic acid, a vitamin C derivative, or combinations thereof, preferably the vitamin C may include the vitamin C derivative, more preferably the vitamin C derivative may include sodium ascorbyl phosphate.

In at least one implementation, the vitamin C may be present in an amount of from about 0.01 wt % to about 5 wt %, preferably about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition.

In at least one implementation, the benefit agent includes the one or more antibacterial agents, wherein the antibacterial agents comprise cetylpyridinium chloride.

In at least one implementation, the orally care composition may include a combination of benzyl alcohol and taurate or a salt thereof, wherein the combination is configured to maintain the cetylpyridinium chloride in an active form.

In at least one implementation, the benzyl alcohol and the taurate or a salt thereof are present in a weight ratio of from about 1:0.2 to about 1:2, about 1:0.5; about 1:1.5, about 1:0.8 about 1:1.1, or about 1:9.

The foregoing and/or other aspects and utilities embodied in the present disclosure may be achieved by providing a method for treating, preventing, or inhibiting dry mouth and/or inflammation of the oral cavity or a surface thereof. The method may include contacting the oral cavity or a surface thereof with any of the oral care compositions disclosed herein.

The foregoing and/or other aspects and utilities embodied in the present disclosure may be achieved by providing a method for improving stability of cetylpyridinium chloride in oral care compositions including hyaluronic acid, the method comprising contacting the oral care compositions including the hyaluronic acid with a combination of benzyl alcohol and taurate or a salt thereof, wherein the combination is configured to maintain the cetylpyridinium chloride in an active form.

In at least one implementation, the benzyl alcohol and the taurate or a salt thereof are present in a weight ratio of from about 1:0.2 to about 1:2, about 1:0.5; about 1:1.5, about 1:0.8 about 1:1.1, or about 1:9.

Further areas of applicability of the present disclosure will become apparent from the detailed description provided hereinafter. It should be understood that the detailed description and specific examples, while indicating some typical aspects of the disclosure, are intended for purposes of illustration only and are not intended to limit the scope of the disclosure.

### DETAILED DESCRIPTION

The following description of various typical aspect(s) is merely exemplary in nature and is in no way intended to limit the disclosure, its application, or uses.

As used throughout this disclosure, ranges are used as shorthand for describing each and every value that is within the range. It should be appreciated and understood that the description in a range format is merely for convenience and brevity, and should not be construed as an inflexible limitation on the scope of any embodiments or implementations disclosed herein. Accordingly, the disclosed range should be construed to have specifically disclosed all the possible subranges as well as individual numerical values within that range. As such, any value within the range may be selected as the terminus of the range. For example, description of a range such as from 1 to 5 should be considered to have specifically disclosed subranges such as from 1.5 to 3, from 1 to 4.5, from 2 to 5, from 3.1 to 5, etc., as well as individual numbers within that range, for example, 1, 2, 3, 3.2, 4, 5, etc. This applies regardless of the breadth of the range.

Unless otherwise specified, all percentages and amounts expressed herein and elsewhere in the specification should be understood to refer to percentages by weight. The amounts given are based on the active weight of the material.

Additionally, all numerical values are “about” or “approximately” the indicated value, and take into account experimental error and variations that would be expected by a person having ordinary skill in the art. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), 1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

As used herein, “free” or “substantially free” of a material may refer to a composition, component, or phase where the material is present in an amount of less than 10.0 weight %, less than 5.0 weight %, less than 3.0 weight %, less than 1.0 weight %, less than 0.1 weight %, less than 0.05 weight %, less than 0.01 weight %, less than 0.005 weight %, or less than 0.0001 weight % based on a total weight of the composition, component, or phase.

All references cited herein are hereby incorporated by reference in their entireties. In the event of a conflict in a definition in the present disclosure and that of a cited reference, the present disclosure controls.

Compositions disclosed herein may be or include an oral care product or an oral care composition thereof. For example, the composition may be an oral care product including the oral care composition and/or one or more additional ingredients/components. In another example, the composition may be the oral care composition of the oral care product. As used herein, the expression “oral care product” may refer to the final form which is sold to a consumer or administered to a user (e.g., patient). The oral care product may be a product that includes one or more chemical compounds or chemical compositions that may be applied to an oral cavity or a surface thereof to therapeutically or non-therapeutically treat a condition (e.g., oral malodor), deliver a benefit agent, improve the health of the user's oral cavity (e.g., vestibule, lips, jaws, palate, teeth, tongue, etc.), or a combination thereof.

The oral care product or the oral care composition thereof may be a liquid, a fluid, a gel, or a paste. Illustrative oral care products or compositions of the present disclosure may be or include, but are not limited to, a toothpaste (dentifrice), an oral rinse, a mouth rinse, a denture cleaner, a saliva substitute, a mouthwash, an oral balm, a serum (e.g., concentrated product), a serum pen, an oral gel, such as a leave-on gel, or any other oral care product intended to contact surfaces of the oral cavity. As used herein, the terms or expressions “mouthwash,” “mouth rinse,” “oral rinse,” or the like, may refer to a liquid that contacts surfaces of the oral cavity or mouth passively or actively. In a preferred implementation, the oral care product or the composition thereof is an oral gel, such as a leave-on oral gel, or an oral balm.

The compositions disclosed herein may be or include oral care compositions including an orally acceptable vehicle or carrier, hyaluronic acid, one or more benefit agents, or combinations thereof. As further described herein, the hyaluronic acid may be capable of or configured to therapeutically or non-therapeutically treat, prevent, or otherwise inhibit one or more conditions of the oral cavity.

As used herein, the term “hyaluronic acid” or “HA” may refer to or include hyaluronic acid, any one or more salts thereof, any one or more hyaluronic acid derivatives, or combinations thereof. Illustrative salts of hyaluronic acid or hyaluronate salts may be or include, but are not limited to, sodium hyaluronate, potassium hyaluronate, magnesium hyaluronate, calcium hyaluronate, or the like, or combinations thereof. Hyaluronic acid is an anionic, non-sulfated glycosaminoglycan (GAG). Hyaluronic is also a naturally occurring mucopolysaccharide found, for example, in synovial fluid, in vitreous humor, in blood vessel walls and umbilical cord, and in other connective tissues. The hyaluronic acid may be or include low-molecular weight hyaluronic acid, middle molecular weight hyaluronic acid, high-molecular weight hyaluronic acid, or combinations thereof. As used herein, the expression “low-molecular weight hyaluronic acid” or “LMW-HA” may refer to hyaluronic acid having a molecular weight of from about 400,000 Daltons (Da) to about 1,000,000 Da. As used herein, the expression “middle molecular weight hyaluronic acid” may refer to hyaluronic acid having a molecular weight of from about 1,000,000 Da to about 1,800,000 Da. As used herein, the expression “high-molecular weight hyaluronic acid” or “HMW-HA” may refer to hyaluronic acid having a molecular weight greater than 1,800,000 Da.

The hyaluronic acid may be present in the oral care composition in an amount effective to treat, prevent, or otherwise inhibit inflammation of the oral cavity. The hyaluronic acid may also be present in the oral care composition in an amount effective to treat, prevent, or otherwise inhibit dry mouth. The hyaluronic acid may be present in the oral care composition in an amount of from about 0.01 wt % to about 10 wt %, based on the total weight of the oral care composition. For example, the hyaluronic acid may be present in an amount of from about 0.01 wt % to about 10 wt %, preferably, about 0.05 wt % to about 5 wt %, more preferably about 0.05 wt % to about 1 wt %, even more preferably about 0.05 wt % to about 0.5 wt %, based on the total weight of the composition. In a preferred implementation, the hyaluronic acid includes sodium hyaluronate in an amount of from about 0.01 wt % to about 5 wt %, about 0.01 wt % to about 1 wt %, about 0.01 wt % to about 0.5 wt %, preferably, about 0.05 wt % to about 0.5%, more preferably about 0.05 wt % to about 0.2 wt %, even more preferably about 0.1 wt %, based on the total weight of the composition.

As used herein, the expression “orally acceptable vehicle” or “carrier” may refer to a suitable vehicle, ingredient, or combination of ingredients, which may be utilized to dissolve, disperse, suspend, hold, mobilize, or otherwise contain the hyaluronic acid and/or the one or more benefit agents. In a preferred implementation, the composition includes the hyaluronic acid and the benefit agents dispersed or otherwise contained in the orally acceptable vehicle or carrier. The orally acceptable vehicle may include one or more thickeners or thickening agents, one or more humectants, one or more solvents, one or more pH modifying agents, one or more flavorants or flavoring agents, one or more preservatives (e.g., natural benzyl alcohol), one or more antioxidants, one or more sweeteners, one or more additional ingredients, or a combination thereof.

The one or more thickeners or thickening agents may be or include one or more polymers capable of or configured to modify (i.e., increase or decrease) the viscosity of the oral care composition. The one or more thickeners or the polymers thereof may be or include, but are not limited to, one or more nonionic thickening polymers, one or more anionic thickening polymers or gelling agents, or combinations thereof. The one or more polymers may be or include water-dispersible or water-soluble hydrophilic colloids.

The one or more polymers of the thickening agents may be or include polysaccharides. The one or more polymers or polysaccharides thereof may be unmodified, as isolated from their source materials, or may be modified as is well known in the polymer art, such as by acetylation, hydroxyalkylation, carboxyalkylation, hydroxyalkylation carboxyalkylation, cationic substitution or a combination thereof. The one or more polymers of the thickening agents may be or include natural and/or modified natural polymers and gums. Illustrative polysaccharides may be or include, but are not limited to, cellulose derivatives (e.g., carboxymethyl cellulose), gums or polysaccharide gums, or a combination thereof. Illustrative gums or polysaccharide gums may be or include, but are not limited to, xanthan gum, carrageenan gum, guar gum, succinoglucan gun, welan gum, gum Arabic, tragacanth gum, locust bean gum, or the like, or a combination thereof.

The anionic polymeric thickeners of the thickening agents may include polyacrylates, such as acrylate-alkyl acrylate copolymers, preferably those selected from carbomers or carbopols (CARBOPOL®, commercially available from Lubrizol Corp. of Wickliffe, OH). Carbomers are homopolymers of acrylic acid crosslinked with an allyl ether of pentaerythritol, sucrose, or propylene. Illustrative acrylate copolymers and/or acrylate-alkyl acrylate copolymers may be or include, but are not limited to, CARBOPOL®) 1382, CARBOPOL®981, CARBOPOL®5984, AQUA SF-1, or the like, or combinations thereof, each of which are commercially available from Lubrizol Corp., or the like, or combinations thereof. Illustrative acrylate copolymers and/or acrylate-alkyl acrylate copolymers may be or include, but are not limited to, those having INCI name acrylates/C10-30 alkyl acrylate crosspolymer, such as CARBOPOL®1382, CARBOPOL®ETD 2020, CARBOPOL®Ultrez 21, PEMULEN TR1, PEMULEN TR2, or the like, or combinations thereof, each of which are commercially available from Lubrizol Corp. The acrylates/C10-30 alkyl acrylate crosspolymers are copolymers of C10-30 alkyl acrylates and one or more monomers of acrylic acid, methacrylic acid or their simple esters thereof crosslinked with an allyl ether of sucrose or pentaerythritol. In a preferred implementation, the one or more thickening agents include a polyacrylate thickener, more preferably, an acrylates/C10-30 alkyl acrylate crosspolymer, even more preferably CARBOPOL®ETD 2020.

It should be appreciated that the acrylates/C10-30 alkyl acrylate crosspolymer may be capable of or configured to facilitate or increase viscosity and/or thickening without heating. The acrylates/C10-30 alkyl acrylate crosspolymer may also be capable of or configured to provide a smooth, sheer thinning texture similar to conventional lip balm, thereby providing a smooth and soothing feeling to soft tissue (e.g., gums, cheeks, tongue, etc.). The acrylates/C10-30 alkyl acrylate crosspolymer may also be capable of or configured to provide a protective barrier on soft tissue or surfaces thereof to prevent, deflect, inhibit microbes (e.g., bacteria) from attaching to the soft tissue. For example, the acrylates/C10-30 alkyl acrylate crosspolymer may promote, provide, or improve barrier integrity as a mucoadhesive polymer. The acrylates/C10-30 alkyl acrylate crosspolymer is an anionic, mucoadhesive polymer that increases the substantivity and film-forming behavior of formulations. A mucoadhesive film of hydrated Carbopol may act as a protective barrier, thereby shielding soft tissue and/or surfaces thereof from bacterial attachment, irritation, environmental stressors (e.g., air pollution, LPS, cigarette smoke, etc.), or combinations thereof.

The one or more thickening agents may be present in an amount effective to sufficiently increase the viscosity of the oral care composition. For example, any one or more of the thickening agents may be present in an amount of from about 0.01 wt % to about 40 wt %, based on the total weight of the composition. In another example, any one or more of the thickening agents may be present in an amount of from about 0.05 wt % to about 10 wt %, based on the total weight of the composition. For example, any one or more of the thickening agents may be present in an amount of from about 0.01 wt % to about 20 wt %, preferably, about 0.05 wt % to about 15 wt %, more preferably about 0.05 wt % to about 10 wt %, even more preferably about 0.1 wt % to about 2 wt t %, or about 1 wt %, based on the total weight of the composition. In a preferred implementation, the one or more thickening agents includes the polyacrylate thickener, more preferably, the acrylates/C10-30 alkyl acrylate crosspolymer, even more preferably CARBOPOL®ETD 2020, wherein the thickening agents are present in an amount of from about 0.01 wt % to about 20 wt %, preferably, about 0.05 wt % to about 15 wt %, more preferably about 0.05 wt % to about 10 wt %, even more preferably about 0.1 wt % to about 2 wt %, or about 1 wt %, based on the total weight of the composition.

As used herein, the term or expression “humectant” may refer to a substance having affinity for water with stabilizing action on the water content of a material. The one or more humectants may include polyols, such as edible polyhydric alcohols (e.g., sugar alcohols). Illustrative humectants may be or include, but are not limited to, glycerin, butylene glycol, propylene glycol, sorbitol, xylitol, polyethylene glycol, or the like, or a combination thereof. In a preferred implementation, the humectants of the oral care composition include xylitol, glycerin, sorbitol, or a combination thereof. For example, the humectants include a combination of xylitol and glycerin.

The one or more humectants may be present in an amount of from 5 weight % to about 80 weight %, based on the total weight of the composition. For example, any one or more of the humectants may be present in an amount of from about 5 weight %, about 15 weight %, about 25 weight %, or about 35 weight % to about 45 weight %, about 55 weight %, about 65 weight %, about 75 weight %, or about 80 weight %, based on the total weight of the composition. In another example, any one or more of the humectants may be present in an amount of from about 5 weight % to about 80 weight %, about 15 weight % to about 75 weight %, about 25 weight % to about 65 weight %, about 35 weight % to about 55 weight %, or about 35 weight % to about 45 weight %, or about 40 wt %, based on the total weight of the composition. In a preferred implementation, the humectants may be present in an amount of about 25 weight % to about 55 weight %, preferably about 30 weight % to about 50 weight %, more preferably about 35 weight % to about 45 weight %, or about 40 weight %, based on the total weight of the composition.

In at least one implementation, the humectants are selected from xylitol, glycerin, or combinations thereof. For example, the humectants may include a combination of xylitol and glycerin. The xylitol may be present in an amount of from about 1 wt % to about 5 wt %, about 2 wt % to about 4 wt %, or about 3 wt %, based on the total weight of the oral care composition. The glycerin may be present in an amount of from about 25 weight % to about 55 weight %, preferably about 30 weight % to about 50 weight %, more preferably about 35 weight % to about 45 weight %, or about 40 weight %, based on the total weight of the composition.

The one or more solvents of the orally acceptable vehicle may be or include any suitable solvent compatible with the remaining components of the oral care product or the oral care composition thereof. Illustrative solvents of the orally acceptable vehicle may be or include, but are not limited to, water, such as purified water or deionized water, ethanol, or the like, or a combination thereof: In a preferred implementation, the orally acceptable vehicle includes water, more preferably demineralized water. Water may make up the balance of the oral care composition or the orally acceptable vehicle thereof. While water may make up the balance of the oral care composition or the orally acceptable vehicle thereof, water may be maintained or present in an amount of less than or equal to 65 wt %, less than or equal to 60 wt %, less than or equal to 55 wt %, less than or equal to 50 wt %, or less than or equal to 45 wt %, based on the total weight of the oral care composition. It should be appreciated that limiting the amount of water may reduce or prevent oxidation of one or more components of the oral care composition. For example, limiting the amount of water may reduce or prevent the oxidation of the one or more flavorants contained in the oral care composition, thereby improving the stability thereof.

The weight ratio of water to any one or more of the humectants may be modified to reduce or prevent oxidation of one or more components of the oral care composition. For example, the weight ratio of water to glycerin may be modified to reduce or prevent the oxidation of the one or more flavorants contained in the oral care composition, thereby improving the stability thereof. The weight ratio of water to glycerin may be less than or equal to 1.6:1, less than or equal to 1.5:1, less than or equal to 1.4:1, less than or equal to 1.3:1, less than or equal to 1.2:1, or less than or equal to 1.1:1.

The one or more pH modifying agents may be or include one or more bases or basifying agents, one or more acids or acidifying agents, one or more buffers or buffering agents, or a combination thereof. The one or more pH modifying agents may be capable of or configured to provide the oral care product or the oral care composition thereof a pH of from 5 to 9, from 5 to 8, from 5 to 8, from 6 to 8, from 7 to 8, or about 7.5. Illustrative pH modifying agent are known in the art and may include, but are not limited to, carboxylic, phosphoric, and sulfonic acids, acid salts (e.g., monosodium citrate, disodium citrate, monosodium malate, etc.), alkali metal hydroxides, such as sodium hydroxide, carbonates, such as sodium carbonate, bicarbonates, sesquicarbonate, borates, silicates, phosphates (e.g., monosodium phosphate, trisodium phosphate, pyrophosphate salts, etc.), or the like, or a combination thereof. It should be appreciated that any one or more of the pH modifying agents may be present in an amount effective to maintain the composition in an orally acceptable pH range. In a preferred implementation, the oral care composition includes sodium hydroxide in an amount effective to at least partially neutralize the one or more thickening agents. For example, the oral care composition may include sodium hydroxide in an amount of from about 0.1 w t % to about 1 wt %, about 0.3 wt % to about 0.8 wt %, or about 0.5 wt % to at least partially neutralize the acrylates/C10-30 alkyl acrylate crosspolymer thickening agent.

The one or more flavorants or flavoring agents may be or include, but are not limited to, sweeteners, flavoring oils or essential oils, sucrose, sucralose, dextrose, polydextrose, dextrin, mannose, xylose, ribose, fructose, levulose, galactose, corn syrup, including high fructose corn syrup and corn syrup solids, partially hydrolyzed starch, lactose, maltose, xylitol, stevia, sodium cyclamate, perillartine, aspartame, liquorice, hydrogenated starch hydrolysate, sorbitol, mannitol, maltitol, isomalt, aspartame, neotame, saccharin and salts thereof (e.g., sodium saccharin), dipeptide-based intense sweeteners, cyclamates, FREEZESTORM®, which is commercially available from Firmenich of Geneva, Switzerland, dihydrochalcones saccharin or a salt thereof, or a combination thereof. Examples of the essential oils include oils of spearmint, peppermint, wintergreen, menthol, sassafras, clove, sage, eucalyptus, marjoram, cinnamon, lemon, lime, grapefruit, and orange. Any one or more of the flavorants or flavoring agents may be present in an amount of from greater than 0 wt % to less than or equal to 1 wt %, less than or equal to 0.8 wt %, less than or equal to 0.7 wt %, less than or equal to 0.6 wt %, less than or equal to 0.5 wt %, less than or equal to 0.4 wt %, less than or equal to 0.35 wt %, less than or equal to 0.30 wt %, less than or equal to 0.25 wt %, less than or equal to 0.20 wt %, less than or equal to 0.1 wt %, or less than or equal to 0.05 wt %.

As used herein, the term or expression “benefit agent” may refer to a substance, compound, material, active, or the like that enhances, promotes, or otherwise provides an advantageous function, a favorable or desirable effect, or benefit. Any one or more of the benefit agents may be present in the oral care composition in an amount effective to enhance, promote, or otherwise provide the advantageous function, the favorable or desirable effect, or benefit. Illustrative benefit agents may be or include, but are not limited to, one or more preservatives (e.g., natural benzyl alcohol), one or more amino acids, one or more zinc ion sources, vitamin C, one or more additional vitamins, curcumin, tetrahydrodiferuloylmethane, clove bud oil or clove oil, Cannabis saliva seed oil or hemp seed oil, one or more prebiotics, one or more antioxidants, one or more sweeteners, one or more allate, collagen, collagen peptides and/or derivatives thereof, phytic acid, sodium phytate, protease, lipase, amylase, glucoamylase, one or more prebiotic agents, one or more antibacterial agents (e.g., cetylpyridinium chloride), one or more sensates, one or more additional ingredients, or a combination thereof.

The one or more amino acids may be or include, any amino acid. Illustrative amino acids may be or include, but are not limited to, common natural amino acids, such as lysine, arginine, histidine, glycine, serine, threonine, asparagine, glutamine, cysteine, selenocysteine, proline, alanine, valine, isoleucine, leucine, methionine, phenylalanine, tyrosine, tryptophan, aspartic acid, glutamic acid, or the like, or combinations thereof. In a preferred implementation, the amino acids include at least glycine. In a preferred implementation, the amino acids are selected from glycine, arginine, or combinations thereof. For example, the amino acids may include glycine and arginine. Any one or more of the amino acids may be present in an amount of from greater than 0 wt % to less than or equal to 3 wt %, less than or equal to 2 wt %, less than or equal to 1.5 wt %, less than or equal to 1 wt %, less than or equal to 0.8 wt %, less than or equal to 0.7 wt %, less than or equal to 0.6 wt %, less than or equal to 0.5 wt %, less than or equal to 0.4 wt %, less than or equal to 0.35 wt %, less than or equal to 0.30 wt %, less than or equal to 0.25 wt %, less than or equal to 0.20 wt %, less than or equal to 0.1 wt %, or less than or equal to 0.05 wt %.

The one or more zinc ion sources of the benefit agents may be capable of or configured to provide one or more zinc ions. The zinc ion source may be or include a chemical compound, a complex, or a zinc salt capable of or configured to provide the zinc ions. The zinc ion source may be capable of or configured to improve oral health. For example, the zinc ion source may be capable of or configured to demineralize/remineralize teeth, prevent or reduce tooth decay, prevent or inhibit disease, such as gingivitis, mild periodontal issues, and other gum diseases, treat or inhibit inflammation, improve immunity, or the like, or any combination thereof. The zinc ion source may also be capable of or configured to promote tissue barrier integrity, improve malodor by reducing, preventing, or inhibiting oral bacteria, inhibit or treat tartar buildup on surfaces of enamel, reduce or inhibit enamel erosion (e.g., erosion from acidic drinks), or combinations thereof. Illustrative zinc ion sources may be or include, but are not limited to, zinc sulfate, zinc chloride, zinc acetate, zinc phenolsulfonate, zinc borate, zinc bromide, zinc nitrate, zinc glycerophosphate, zinc benzoate, zinc carbonate, zinc citrate (e.g., zinc citrate trihydrate), zinc hexafluorosilicate, zinc phosphate hydrate, zinc lactate trihydrate, zinc oxide, zinc peroxide, zinc salicylate, zinc silicate, zinc stannate, zinc tannate, zinc tartrate, zinc titanate, zinc tetrafluoroborate, or a combination thereof. In a preferred implementation, the zinc ion source includes zinc phosphate hydrate. Any one or more of the zinc ion sources may be present in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

As used herein, the term or expression “vitamin C” includes vitamin C (i.e., ascorbic acid), vitamin C derivatives, or combinations thereof. Vitamin C derivatives may be or include compounds or substances that release vitamin C in vivo or in vitro, solvates, hydrates, and salts thereof. Illustrative vitamin C derivatives may be or include, but are not limited to, glucosides of ascorbic acid or ascorbyl glucoside (ASG), phosphates of ascorbic acid, in particular magnesium ascorbyl phosphate, sodium ascorbyl phosphate, calcium ascorbyl phosphate, potassium ascorbyl phosphate, mixed salts, such as sodium magnesium ascorbyl phosphate or sodium calcium ascorbyl phosphate, or the like, or a combination thereof. It should be appreciated that the phosphates may often exist as hydrates, where the dihydrate form is the most common. In a preferred implementation, the vitamin C includes sodium ascorbyl phosphate.

The Vitamin C may be capable of or configured to inhibit, prevent, reduce, or treat inflammation and/or oxidative stress, promote host immunity, promote healthy soft tissue (e.g., gum, cheeks, tongue, etc.) in the oral cavity, or combinations thereof. The Vitamin C may be present in the oral care composition in an amount effective to inhibit, prevent, reduce, or treat inflammation and/or oxidative stress, and/or promote host immunity. For example, the Vitamin C may be present in the oral care composition in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

It should be appreciated that other vitamins may also be provided. For example, the oral care composition may also include Vitamin D and E. The other vitamins may be capable of or configured to promote gum health. Any one or more of the other vitamins may be present in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

The tetrahydrodiferuloylmethane may be capable of or configured to act as an antioxidant and/or anti-inflammatory agent for soft tissues of the oral cavity to thereby promote healthy guns. The tetrahydrodiferuloylmethane may be present in the oral care composition in an amount effective to act as an antioxidant and/or anti-inflammatory agent for soft tissues of the oral cavity to thereby promote healthy gums. For example, the tetrahydrodiferuloylmethane may be present in the oral care composition in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

The clove oil may be capable of or configured to provide one or more of pain relief, flavoring properties (as discussed above), anesthetic effects, antimicrobial properties, antibacterial, antifungal, anti-inflammatory, pain-relief, soothing relief, or combinations thereof. The clove oil may be present in the oral care composition in an amount effective to provide one or more of the following: pain relief, flavoring properties (as discussed above), anesthetic effects, antimicrobial properties, antibacterial, antifungal, anti-inflammatory, pain-relief, soothing relief, or combinations thereof. For example, the clove oil may be present in the oral care composition in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

The hemp seed oil may be capable of or configured to provide one or more of the following properties or functions: soothing, antibacterial, anti-fungal, anti-inflammatory, antioxidant, or combinations thereof. The hemp seed oil may be present in the oral care composition in an amount effective to provide one or more of the following properties or functions: soothing, antibacterial, anti-fungal, anti-inflammatory, antioxidant, or combinations thereof. For example, the hemp seed oil may be present in the oral care composition in an amount of from about 0.01 wt % to about 5 wt %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition or the orally acceptable vehicle thereof.

The one or more sensates may be capable of or configured to provide one or more of the following properties or functions: reduce discomfort, impart a mild tingling sensation, impart a cooling sensation, impart a numbing sensation, reduce sensitivity, or a combination thereof. Illustrative sensates may be or include, but are not limited to, one or more of the flavorants disclosed herein, such as FREEZESTORM®, clove bud oil, benzyl alcohol, methanol, or a combination thereof. In one implementation, the sensates include a combination of clove bud oil, benzyl alcohol, and one or more flavorants. In an exemplary implementation, the sensates include a combination of clove bud oil, benzyl alcohol, and FREEZESTORM®.

In at least one implementation, the oral care composition or product disclosed herein may include one or more antibacterial agents capable of or configured to inhibit, reduce, treat, and/or prevent bacteria and other microorganisms. The one or more antibacterial agents may also be capable of or configured to inhibit, treat, or otherwise prevent dental plaque and/or gingivitis. In an exemplary implementation, the one or more antibacterial agents may be or include cetylpyridinium chloride. It should be appreciated that cetylpyridinium chloride (CPC) is unstable in combination with or in the presence of hyaluronic acid. Without being bound by theory, it is believed that the CPC and the hyaluronic acid form a complex with one another, thereby resulting in a decrease in available or active CPC and/or hyaluronic acid. The inventors have surprisingly and unexpectedly discovered that the combination of taurate or a salt thereof and benzyl alcohol, in effective amounts, aids or facilitates maintaining the CPC and/or hyaluronic acid in their respective active forms. Without being bound by theory, it is believed that the combination of taurate or a salt thereof and benzyl alcohol increases the respective solubility of CPC and prevents it from forming complexes with other components of the oral care composition, such as hyaluronic acid.

The oral care compositions disclosed herein may not exhibit significant or substantial reduction in the concentration of active CPC after exposure to accelerated aging conditions. The oral care compositions disclosed herein may not exhibit significant or substantial reduction in the concentration of active CPC after exposing the oral care composition to one or more freeze-thaw treatments. For example, the oral care composition disclosed herein may not exhibit significant or substantial decrease in CPC after freeze-thawing treatment between about −30° C., about −25° C., or about −20° (C and about room temperature or about 30° C. for at least or about 2, 3, 4, or more cycles/treatments. As used herein, a significant or substantial reduction in the concentration of active CPC may be greater than a 6% reduction in active CPC, greater than 8% reduction in active CPC, greater than a 10% reduction in active CPC, or more, as compared to the initial concentration of CPC.

The effective amount of benzyl alcohol and taurate for stabilizing or preserving the active CPC may vary. In at least one example, the amount of benzyl alcohol may be present in an amount of from greater than 0 wt % to about 2 wt %, based on the total weight of the oral care composition. For example, the amount of the benzyl alcohol may be from about 0.1 wt % to about 2 wt %, about 0.2 wt % to about wt %, about 0.3 wt % to about 0.8 wt %, or about 0.4 wt % to about 0.6 wt %, or about 0.5 wt %, based on the total weight of the oral care composition. In at least one example, the amount of taurate or a salt thereof may be present in an amount of from greater than 0 wt % to about 2 wt %, based on the total weight of the oral care composition. For example, the amount of taurate or a salt thereof may be from about 0.1 wt % to about 2 wt %, about 0.2 wt % to about 1 wt %, about 0.3 wt % to about 0.8 wt %, or about 0.4 wt % to about 0.6 wt %, or about 0.45 wt %, based on the total weight of the oral care composition. In an exemplary implementation, the effective weight ratio of the benzyl alcohol to the taurate may from about 1:0.2 to about 1:2, about 1:0.5; about 1:1.5, about 1:0.8 about 1:1.1, or about 1:9.

In at least one implementation, the oral care composition or product disclosed herein may include an orally acceptable vehicle or carrier, hyaluronic acid, one or more benefit agents, or combinations thereof. The one or more benefit agents include at least one or more sensates, one or more zinc ion sources, Vitamin C, tetrahydrodiferuloylmethane, or a combination thereof. The one or more sensates may include a combination of clove bud oil, benzyl alcohol, and one or more flavorants. The combination of the sensates may interact synergistically to provide comparable or improved effects compared to relatively higher concentrations of clove bud oil alone. For example, the oral care compositions including the combination of clove bud oil, benzyl alcohol, and one or more flavorants may synergistically provide reduced discomfort, impart a mild tingling sensation, impart a cooling sensation, impart a numbing sensation, reduce sensitivity, or a combination thereof, as compared to a conventional oral care composition including relatively higher concentrations (i.e., greater than 0.15 wt %, greater than 0.25 wt %, greater than 0.3 wt %, or greater than 0.5 wt %) of clove bud oil.

In an exemplary implementation, the combination of sensates may include clove bud oil in an amount greater than 0 wt % to about 0.3 wt %, about 0.05 wt % to about 0.2 wt %, or about 0.05 wt % to about 0.15 wt %, benzyl alcohol in an amount of from 0.2 wt % to 1.5 wt %, about 0.4 wt % to about 1.2 wt %, or about 0.7 wt % to about 0.9 wt %, and one or more flavorants (e.g., FREEZESTORM®) in an amount of from about 0.01 wt % to 2 wt %, about 0.05 wt % to about 1.8 wt %, or about 0.1 wt % to about 1.5 wt %, based on the total weight of the oral care composition.

It should be appreciated by one having ordinary skill in the art, that the oral care products and/or the oral care composition thereof may include other additional ingredients/components. For example, the oral care products and/or the oral care composition thereof may include any one or more of the following: anti-caries agents, diluents, surface active agents or surfactants, mouth feel agents, sweetening agents, colorants or coloring agents, preservatives, antifoam agents (e.g., benzoic acid, sulfuric acid, glyceryl monostearate, etc.), or the like, or a combination thereof. It should further be appreciated by one having ordinary skill in the art that while general attributes of each of the above categories of materials may differ, there may be some common attributes and any given material may serve multiple purposes within two or more of such categories of materials. For example, clove oil may be capable of or configured to provide a flavor property as well as a therapeutic function.

The present disclosure may provide a method for preparing an oral care product or an oral care composition thereof. The method may include mixing, stirring, combining, or otherwise contacting an orally acceptable vehicle or carrier, hyaluronic acid, one or more benefit agents, or combinations thereof with one another. For example the method may include contacting the carrier, hyaluronic acid, and at least one benefit agent with one another. The method may include contacting the carrier, hyaluronic acid, and at least one benefit agent in respective amount to provide a gel or a balm.

The present disclosure may further provide method for treating, preventing, or otherwise inhibiting inflammation of the oral cavity or a surface thereof. The inflammation may be the result of one or more diseases or conditions. The method may include contacting the oral cavity or a surface thereof with any one or more of the oral care compositions disclosed herein. The method may also include decreasing and/or detecting a decrease in the amount of one or more biomarkers for inflammation. The one or more biomarkers for inflammation may be or include, but are not limited to, Interleukin-8 (IL-8), prostaglandin E2 (PGE2), or combinations thereof. The method may also include diagnosing or determining the presence of oral inflammation or a condition or disease that may cause oral inflammation. The method for diagnosing or determining the presence of oral inflammation or a condition/disease that may cause oral inflammation may include measuring an elevated amount of one or more biomarkers for inflammation relative to a population baseline value or a previous individual baseline value. It should be appreciated that the population baseline value may be the value from a population that does not have or exhibit oral inflammation or a condition/disease that may cause oral inflammation. The elevated amount of the one or more biomarkers may be at least 1% greater, at least 2% greater, at least 5% greater, at least 8% greater, at least 10% greater, at least 12% greater, at least 15% greater, at least 20% greater, at least 25% greater, at least 30% greater, at least 40% greater, at least 50% greater, at least 60% greater, at least 70% greater, at least 80% greater, at least 90% greater, at least 100% greater, at least 150% greater, at least 200% greater, at least 250% greater, at least 300% greater, at least 400% greater, or at least 500% greater than the population or previous individual baseline value.

The present disclosure may further provide method for treating, preventing, or otherwise inhibiting dry mouth. The dry mouth may be the result of one or more diseases or conditions. The dry mouth may also be the result of or a side effect of a medical treatment and/or medication. The method may include contacting the oral cavity or a surface thereof with any one or more of the oral care compositions disclosed herein. The method may include causing the salivary glands to increase production of saliva.

The present disclosure may also provide a method for improving oral care by treating, preventing, or otherwise inhibiting discomfort, irritation, or pain of the oral cavity or a surface thereof, such as the gums. The present disclosure may also provide a method for improving oral care by soothing the oral cavity or a surface thereof (e.g., gums). For example, oral inflammation, such as periodontitis, gingivitis, and other oral conditions, often result in increased irritation and/or sensitivity of the gums. The increased irritation and/or sensitivity of the gums often resulted in poor tooth brushing to avoid discomfort in sensitive and/or irritated areas of the oral cavity, which leads to increased oral inflammation and disease. The method may include contacting the oral cavity or a surface thereof with any one or more of the oral care compositions or oral care products disclosed herein to treat, prevent, or otherwise inhibit discomfort, irritation, or gum pain. The method may also include contacting the oral cavity or a surface thereof with any one or more of the oral care compositions or oral care products disclosed herein to sooth the oral cavity or a surface thereof. Contacting the oral cavity or a surface thereof with the oral care compositions or products disclosed herein may impart a tingling, cooling, and/or numbing effect that facilitates the reduction in discomfort associated with brushing and/or flossing. The oral care compositions or oral care products may be applied before brushing and/or flossing to reduce the discomfort thereof. The oral care compositions or oral care products may also be applied after brushing and/or flossing to sooth the discomfort thereof.

The present disclosure may also provide a method for stabilizing one or more antibacterial agents, such as cetylpyridinium chloride, in oral care compositions including hyaluronic acid. The method for stabilizing the one or more antibacterial agents may include contacting the oral care composition including hyaluronic acid with effective amounts of benzyl alcohol and taurate or a salt thereof. The benzyl alcohol and taurate or a salt thereof may be present in an effective amount to improve solubility of the CPC and/or prevent the formation of complexes with other ingredients, such as hyaluronic acid.

The following numbered paragraphs disclose one or more exemplary variations of the subject matter of the application:

1. An oral care composition, comprising: an orally acceptable vehicle; hyaluronic acid; and one or more benefit agents.

2. The oral care composition of paragraph 1, wherein the hyaluronic acid comprises one or more of hyaluronic acid, a salt of hyaluronic acid, a derivative of hyaluronic acid, or combinations thereof, preferably, the hyaluronic acid comprises a salt of hyaluronic acid, more preferably, the salt of hyaluronic acid comprises sodium hyaluronate.

3. The oral care composition of paragraphs 1 or 2, wherein the hyaluronic acid is present in an amount effective to treat, prevent, or inhibit inflammation of the oral cavity, preferably, the hyaluronic acid is present in an amount of from about 0.05 wt % to about 0.5%, preferably, about 0.05 wt % to about 0.2 wt %, more preferably about 0.1 wt %, based on the total weight of the composition.

4. The oral care composition of any one or more of paragraphs 1 to 3, wherein the hyaluronic acid comprises one or more of low-molecular weight hyaluronic acid, middle molecular weight hyaluronic acid, high-molecular weight hyaluronic acid, or combinations thereof, preferably, the hyaluronic acid comprises at least low-molecular weight hyaluronic acid.

5. The oral care composition of any one or more of paragraphs 1 to 4, wherein the orally acceptable vehicle comprises one or more of a thickening agent, a humectant, a solvent, a pH modifying agent, a flavorant, or combinations thereof, preferably, the orally acceptable vehicle comprises a combination of the thickening agent, the humectant, the solvent, the pH modifying agent, and the flavorant.

6. The oral care composition of paragraph 5, wherein the thickening agent comprises an anionic polymeric thickener, preferably, an acrylate copolymer, an acrylate-alkyl acrylate copolymer, or combinations thereof, more preferably the thickening agent comprises an acrylates/C10-30 alkyl acrylate crosspolymer.

7. The oral care composition of any one or more of paragraphs 5 or 6, wherein the thickening agent is present in an amount of from about 0.01 wt % to about 30 wt %, preferably, about 0.05 wt % to about 15 wt %, more preferably about 0.1 wt % to about 5 wt %, even more preferably about 0.1 wt % to about 2 wt %, or about 1 wt %, based on the total weight of the composition.

8. The oral care composition of any one or more of paragraphs 5 to 7, wherein the humectant comprise one or more polyols, preferably, the humectant comprises one or more of xylitol, glycerin, or combinations thereof, more preferably the humectant comprises a combination of xylitol and glycerin.

9. The oral care composition of any one or more of paragraphs 5 to 8, wherein the humectant is present in an amount of from about 25 weight % to about 55 weight %, preferably about 30 weight % to about 50 weight %, more preferably about 35 weight % to about 45 weight %, or about 40 weight %, based on the total weight of the composition.

10. The oral care composition of paragraphs 8 or 9, wherein the solvent comprises water, preferably, a weight ratio of water to glycerin is less than or equal to 1.6:1, less than or equal to 1.5:1, less than or equal to 1.4:1, less than or equal to 1.3:1, less than or equal to 1.2:1, or less than or equal to 1.1:1.

11. The oral care composition of any one or more of paragraphs 1 to 10, wherein the benefit agents comprise one or more of a zinc source, vitamin C, tetrahydrodiferuloylmethane, clove oil, hemp seed oil, one or more amino acids, or combinations thereof, preferably the one or more amino acids is selected from arginine, glycine, or combinations thereof.

12. The oral care composition of paragraph 11, wherein the benefit agent comprises the zinc source, preferably, the zinc source comprises zinc phosphate hydrate, more preferably, the zinc source is present in an amount of from about 0.01 wt % to about 5 w t %, preferably from about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition.

13. The oral care composition of paragraphs 11 or 12, wherein the benefit agent comprises vitamin C, wherein vitamin C comprises one or more of ascorbic acid, a vitamin C derivative, or combinations thereof, preferably the vitamin C comprises the vitamin C derivative, more preferably the vitamin C derivative comprises sodium ascorbyl phosphate.

14. The oral care composition of paragraphs 11 to 13, wherein the vitamin C is present in an amount of from about 0.01 wt % to about 5 wt %, preferably about 0.05 wt % to about 1 wt %, more preferably about 0.1 wt % to about 0.5 wt %, even more preferably about 0.1 wt % to about 0.3 wt %, or about 0.2 wt %, based on the total weight of the oral care composition.

15. The oral care composition of paragraphs 11 to 14, wherein the benefit agent comprises the one or more antibacterial agents, wherein the antibacterial agents comprise cetylpyridinium chloride.

16. The oral care composition of paragraphs 15, further comprising a combination of benzyl alcohol and taurate or a salt thereof, wherein the combination is configured to maintain the cetylpyridinium chloride in an active form.

17. The oral care composition of paragraphs 16, wherein the benzyl alcohol and the taurate or a salt thereof are present in a weight ratio of from about 1:0.2 to about 1:2, about 1:0.5; about 1:1.5, about 1:0.8 about 1:1.1, or about 1:9.

18. A method for treating, preventing, or inhibiting dry mouth, inflammation, and/or irritation or discomfort of the oral cavity or a surface thereof, the method comprising contacting the oral cavity or the surface thereof with the oral care composition of any of paragraphs 1 to 17.

19. A method for improving stability of cetylpyridinium chloride in oral care compositions including hyaluronic acid, the method comprising contacting the oral care compositions including the hyaluronic acid with a combination of benzyl alcohol and taurate or a salt thereof, wherein the combination is configured to maintain the cetylpyridinium chloride in an active form.

20. The method of paragraphs 19, wherein the benzyl alcohol and the taurate or a salt thereof are present in a weight ratio of from about 1:0.2 to about 1:2, about 1:0.5; about 1:1.5, about 1:0.8 about 1:1.1, or about 1:9.

All ingredients for use in the compositions described herein should be orally acceptable. As used herein, “orally acceptable” may refer any ingredient that is present in a composition as described in an amount and form which does not render the composition unsafe for use in the oral cavity.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

The stability of six oral care compositions (1)-(6) incorporating hyaluronic acid were evaluated. Particularly, the oral care compositions (1)-(6) were evaluated for their respective stability with respect to coloring (e.g., yellowing), flavor delivery, physical stability (e.g., separation), and microbiology or microbiological stability as it relates to the resistance of the composition to bacterial growth. The six oral care composition (1)-(6) were prepared by combining the ingredients/components according to Table 1.

TABLE 1
Oral Care Compositions (1)-(6)

| Ingredient/ | 1 | 2 | 3 | 4 | 5 | 6 |
|---|---|---|---|---|---|---|
| Component | (wt %) | (wt %) | (wt %) | (wt %) | (wt %) | (wt %) |
| Demineralized | 68.40 | 48.40 | 74.73 | 74.75 | 74.70 | 54.70 |
| Water | | | | | | |
| Glycerin | 10.0 | 30.0 | 20.00 | 20.00 | 20.00 | 40.0 |
| Anionic | 0.80 | 0.80 | 0.80 | 0.80 | 0.80 | 0.80 |
| Polymeric | | | | | | |
| Thickening | | | | | | |
| Agent | | | | | | |
| 50% Sodium | 0.50 | 0.50 | 0.50 | 0.50 | 0.50 | 0.50 |
| Hydroxide | | | | | | |
| Sodium | 0.05 | 0.05 | 0.10 | 0.10 | 0.10 | 0.10 |
| Hyaluronate | | | | | | |
| White | 20.0 | 20.0 | — | — | — | — |
| Petrolatum | | | | | | |
| Xylitol | — | — | 3.0 | 3.0 | 3.0 | 3.0 |
| Preservative | — | — | 0.50 | 0.50 | 0.50 | 0.50 |
| Benefit Agent | — | — | 0.100 | 0.1 | 0.10 | 0.10 |
| Flavorant | 0.25 | 0.25 | 0.27 | 0.25 | 0.3 | 0.3 |
| Total | 100 | 100 | 100 | 100 | 100 | 100 |
| Components | | | | | | |

As indicated in Table 1, oral care compositions (1) and (2) incorporated white petrolatum while oral care compositions (3)-(6) excluded white petrolatum. As further indicated in Table 1, oral care compositions (3)-(6) further included flavorants, sweeteners, and preservatives.

To evaluate the stability of each of the oral care compositions (1)-(6), each of the oral care compositions (1)-(6) was exposed to accelerated aging conditions. Specifically, each of the oral care compositions (1)-(6) were aged for about 13 weeks at 30° C. at 65% relative humidity and/or 40° C. at about 75% relative humidity.

The coloring and physical stabilities of each of the oral care compositions (1)-(6) upon accelerated aging were evaluated visually. The flavor delivery or retention of the flavor upon accelerated aging was evaluated with an expert panel as either pass or fail. Flavor evaluation was conducted on samples aged for about 6 weeks at about 49° C.

It was observed that the weight ratio or relative amounts of water and glycerin was related to the flavor stability of each of the oral care compositions (1)-(6). Particularly, it was surprisingly and unexpectedly discovered that increased amounts of water or an increased weight ratio of water to glycerin, as observed in oral care compositions (1) and (3)-(5), resulted in reduced flavor stability as compared to oral care composition (6). Without being bound by theory, it is believed that the relatively greater amount of water in excess of about 60 wt % resulted in increased oxidation of the flavorant, thereby resulting in decreased flavor delivery and/or retention upon exposure to accelerated aging.

### Example 2

The stability of hyaluronic acid or a salt thereof in an exemplary oral care composition upon exposure to accelerated aging conditions was evaluated. Particularly, the ability of the oral care compositions to retain and stabilize hyaluronic acid upon exposure to accelerated aging conditions was evaluated with a commercially available enzyme-linked immunosorbent assay (ELISA) kit. The oral care composition (3) prepared in Example 1 was utilized as the test oral care composition. A control oral care composition (7) excluding hyaluronic acid was prepared for comparison. Statistical significance was determined at a 90% confidence level via two-tail analysis, and the Tukey method was utilized for grouping and detecting significant differences. The results are summarized in Table 2.

TABLE 2
Quantification of Sodium Hyaluronate by ELISA

| | Sodium Hyaluronate | p vs. |
|---|---|---|
| Sample | Measured (% w/w) | Control* |
| (3) | 0.087 ± 0.011 | <0.00001 |
| Oral Care Composition | | |
| (0.1 wt % HA) | | |
| (7) | 0.001 ± 0.000 | — |
| Control Oral Care | | |
| Composition (0 wt % HA) | | |

As indicated in Table 2, the oral care composition demonstrated the ability to retain and stabilize hyaluronic acid upon exposure to accelerated aging conditions. As such, minimal to no degradation of the hyaluronic acid and/or reduced efficacy of the hyaluronic acid is expected in the oral care compositions disclosed herein.

### Example 3

The efficacy of an exemplary oral care composition (8) including hyaluronic acid for reducing or inhibiting inflammation or its anti-inflammatory ability was evaluated. Specifically, an in vitro study was conducted to observe inflammatory biomarker Interleukin-8 (IL-8) on HEK-hTLR4 cells stimulated for an inflammatory response. The oral care composition (8) was prepared by combining the components according to Table 3.

TABLE 3
Oral Care Composition (8)

| | (8) |
|---|---|
| Ingredient/Component | (wt %) |
| Demineralized Water | 54.4 |
| Glycerin | 40.0 |
| Anionic Polymeric Thickening Agent | 0.80 |
| 50% Sodium Hydroxide | 0.50 |
| Sodium Hyaluronate | 0.1 |
| Xylitol | 3 |
| Preservative | 0.5 |
| Flavorant | 0.5 |
| Glycine | 0.1 |
| Excipients | Balance |
| Total Components | 100 |

To evaluate the efficacy of the oral care composition (8) in the HEK-hTLR4 cell lines, the cells were grown until confluence at 37° C. and 5% CO₂. The cells were then co-incubated overnight with the oral care composition (8) (at a 25× dilution) and about 1 μg/mL of a TLR4 ligand to stimulate an inflammatory response. The TLR4 ligand utilized was an ultrapure lipopolysaccharide from Porphyromonas gingivalis, which is commercially available from Invivogen as tlrl-ppglps. A culture including the TLR4 ligand without the oral care composition (8) was also incubated overnight as a positive control, and an untreated culture was maintained as a negative control.

Inflammation was evaluated by observing the production of IL-8 from the HEK-hTLR4 cells. To observe the production, IL-8 analysis was performed using the IL-8 ELISA kit, commercially available from Enzo Life Sciences Inc. The results are summarized in Table 4.

In addition to evaluating the production of IL-8, cell viability was also analyzed with PrestoBlue™ Cell Viability Reagent, commercially available from Invitrogen (Catalog Number: A13262). The results of the cell viability are also summarized in Table 4.

TABLE 4
Analysis of HEK-hTLR4 Cells

| | IL-8 Production | | Cell Viability | |
|---|---|---|---|---|
| | Average IL-8 | | Average | |
| | Concentration | Std | Viability | Std |
| | (pg/mL) | Dev. | (%) | Dev. |
| Negative Control | 47.57 | 4.20 | 100 | 5.53 |
| Positive Control | 3571.71 | 80.21 | 86.94 | 7.86 |
| Treated with Oral | 73.59 | 3.96 | 55.22 | 3.93 |
| Care | | | | |
| Composition (8) | | | | |

As indicated in Table 4, the positive control, which was incubated without the oral care composition (8) exhibited significant IL-8, indicating inflammation. The negative control, which was not stimulated for an inflammatory response exhibited relatively low concentrations of IL-8. The cells co-incubated with the TLR4 ligand and the oral care composition (8) exhibited IL-8 concentrations similar to the negative control, thereby indicating the efficacy of the oral care composition (8) for inhibiting inflammation. Table 4 further demonstrated that the oral care composition (8) was relatively gentle or mild on HEK cells.

### Example 4

The efficacy of the exemplary oral care composition (8) of Example 3 for reducing or inhibiting inflammation was evaluated. Specifically, an in vitro study was conducted to observe inflammatory biomarker prostaglandin E2 (PGE2) on primary human gingival fibroblasts (hGF) cells stimulated for an inflammatory response.

To evaluate the efficacy in the hGF cell lines, the cells were grown until confluence at 37° C. and 5% CO₂. The cells were then co-incubated overnight with the oral care composition (8) (25× dilution) and about 1 ng/mL of Interleukin-1β (IL-1β)), which is commercially available from Invivogen as rcyec-hillb. A culture including the IL-1β without the oral care composition (8) was also incubated overnight as a positive control, and an untreated culture was maintained as a negative control.

Inflammation was evaluated by observing the production of PGE2 from the hGF cells. To observe the production, PGE2 analysis was performed using the PGE2 ELISA kit, commercially available from Enzo Life Sciences, Inc. The results are summarized in Table 5.

In addition to evaluating the production of IL-8, cell viability was also analyzed with PrestoBlue™ Cell Viability Reagent, commercially available from Invitrogen (Catalog Number: A13262). The results of the cell viability are also summarized in Table 5.

TABLE 5
Analysis of hGF Cells

| | PGE2 Production | | Cell Viability | |
|---|---|---|---|---|
| | Average PGE2 | | Average | |
| | Concentration | Std | Viability | Std |
| | (pg/mL) | Dev. | (%) | Dev. |
| Negative Control | 111.24 | 15.81 | 100 | 8.77 |
| Positive Control | 2687.28 | 38.81 | 89.39 | 9.20 |
| Treated with Oral | 430.59 | 70.68 | 82.08 | 3.08 |
| Care | | | | |
| Composition (8) | | | | |

As indicated in Table 5, the positive control, which was incubated without the oral care composition (8) exhibited significant PGE2, indicating inflammation. The negative control, which was not stimulated for an inflammatory response exhibited relatively low concentrations of PGE2. The cells co-incubated with the IL-1β and the oral care composition (8) exhibited PGE2 concentrations similar to the negative control, thereby indicating the efficacy of the oral care composition (8) for inhibiting inflammation. Table 5 further demonstrated that the oral care composition (8) was relatively gentle or mild on human gingival fibroblast cells.

### Example 5

Oral care composition (9) was prepared by combining the ingredients/components according to Table 6. The oral care composition (9) was similar to the oral care composition (8) of Example 3, but farther included a combination of clove bud oil, FREEZESTORM®, and benzyl alcohol as “sensates”. The sensates or combination of clove bud oil, FREEZESTORM®, and benzyl alcohol provided a mild tingling, cooling, and numbing feel that reduced discomfort during oral care, including brushing. It should be appreciated that the benzyl alcohol contributed as both a sensate as well as a preservative. In addition to the combination of sensates, oral care composition (9) further included additional benefit agents, namely, zinc phosphate, Vitamin C, and curcumin or tetrahydrodiferuloylmethane for improving one or more oral health benefits.

TABLE 6
Oral Care Composition (9)

| | (9) |
|---|---|
| Ingredient/Component | (wt %) |
| Demineralized Water | 53.4 |
| Glycerin | 40.0 |
| Anionic Polymeric Thickening Agent | 0.80 |
| 50% Sodium Hydroxide | 0.50 |
| Sodium Hyaluronate | 0.10 |
| Xylitol | 3 |
| Zinc Phosphate, Hydrate | 0.187 |
| Tetrahydrodiferuloylmethane | 0.15 |
| Benzyl Alcohol | 0.8 |
| Clove Bud Oil | 0.15 |
| Freezestorm ® | 0.05 |
| Excipients | Balance |
| Total Components | 100 |

Oral care composition (9) was evaluated as a pre-brushing treatment. Without being bound by theory, it is believed that the combination of sensates in the oral care composition (9) reduces the amount of clove bud oil necessary to provide substantially the same or the same benefits of the clove bud oil alone but in relatively higher concentrations, as utilized in conventional oral care compositions. The ability to reduce the amount of clove bud oil in oral care compositions thereby provides the same or similar benefits without exceeding regionally regulated limitations for the use of clove bud oil and/or reduces the taste associated with the use of clove bud oil, which may be off-putting for consumers.

### Example 6

The efficacy of exemplary oral care compositions (10)-(12) for reducing or inhibiting inflammation or its anti-inflammatory ability was evaluated. Specifically, an in vitro study was conducted to observe inflammatory biomarker prostaglandin E2 (PGE2) on primary human gingival fibroblasts (hGF) cells stimulated for an inflammatory response. The oral care compositions (10)-(12) were prepared by combining the components according to Table 7.

TABLE 7
Oral Care Compositions (10)-(12)

| | (10) | (11) | (12) |
|---|---|---|---|
| Ingredient/Component | (wt %) | (wt %) | (wt %) |
| Demineralized Water | 54.3 | 54.4 | 54.0 |
| Glycerin | 40 | 40.2 | 40 |
| Anionic Polymeric Thickening Agent | 0.8 | 0.8 | 0.8 |
| 50% Sodium Hydroxide | 0.5 | 0.5 | 0.5 |
| Sodium Hyaluronate | 0.1 | 0.1 | 0.1 |
| Xylitol | 3 | 3 | 3 |
| Preservative | 0.5 | 0.5 | 0.5 |
| Hemp Seed Oil | 0.1 | 0.1 | 0.1 |
| Zinc Phosphate | — | 0.187 | 0.187 |
| Sodium Ascorbyl Phosphate (Vitamin C) | — | 0.1 | 0.1 |
| Curcumin (tetrahydrodiferuloylmethane) | — | — | 0.15 |
| Excipients | Bal. | Bal. | Bal. |
| Total Components | 100 | 100 | 100 |

To evaluate the efficacy of the oral care compositions (10)-(12) in the hGF cell lines, the cells were grown until confluence at 37° C. and 5% CO₂. The cells were then co-incubated overnight with the oral care compositions (10)-(12) (25× dilution) and about 1 ng/mL of Interleukin-1β (IL-1β), which is commercially available from Invivogen as rcyec-hillb. A culture including the IL-1β without any of the oral care compositions (10)-(12) was also incubated overnight as a positive control, and an untreated culture was maintained as a negative control.

Inflammation was evaluated by observing the production of PGE2 from the hGF cells. To observe the production, PGE2 analysis was performed using the PGE2 ELISA kit, commercially available from Enzo Life Sciences, Inc. The results are summarized in Table 8.

TABLE 8
Analysis of hGF Cells

| | PGE2 Production | | |
|---|---|---|---|
| | (pg/mL) | Std Dev. | P value |
| Negative Control | 210.86 | 17.06 | — |
| Positive Control | 5166.98 | 8.19 | — |
| Treated with Oral Care | 3657.21 | 316.08 | 0.014 |
| Composition (10) | | | |
| Treated with Oral Care | 2803.58 | 642.90 | 0.006 |
| Composition (11) | | | |
| Treated with Oral Care | 1289.90 | 67.54 | 0.006 |
| Composition (12) | | | |

As indicated in Table 8, the positive control, which was incubated without the oral care compositions (10)(12) exhibited significant PGE2, indicating inflammation. The negative control, which was not stimulated for an inflammatory response exhibited relatively low concentrations of PGE2. The cells co-incubated with the IL-1β and the respective oral care compositions (10)-(12) all surprisingly and unexpectedly exhibited PGE2 concentrations substantially and significantly lower than the positive control, thereby demonstrating the efficacy of each of the oral care compositions (10)-(12) for inhibiting inflammation.

In addition to evaluating the production of IL-8, cell viability for each of the oral care compositions (10)-(12) was also analyzed with PrestoBlue™ Cell Viability Reagent, commercially available from Invitrogen (Catalog Number: A 3262). Cell viability for each of the oral care compositions (10)-(12) was between 64% and 72% in 3× to 24× diluted solutions of each of the oral care compositions (10)-(12). Accordingly, each of the oral care compositions (10)-(12) was also demonstrated as being relatively gentle or mild on HEK cells.

### Example 7

The stability of CPC in oral care compositions including hyaluronic acid was evaluated. Particularly, oral care compositions (13)-(21) including CPC and hyaluronic acid was evaluated in the presence of varying amounts of benzyl alcohol and/or sodium methyl cocoyl taurate. The oral care compositions (13)-(21) were prepared by combining the components according to Tables 9a and 9b.

To evaluate the stability of CPC, each of the oral care compositions (13)(21) was placed in a respective glass jar and exposed to accelerated aging conditions. Particularly, each of the oral care compositions (13)-(21) was exposed to freeze-thaw conditions between −30° C. and 30° C. for 3 cycles. After exposing each of the oral care compositions (13)-(21) to the aging conditions, the amount of CPC was measured analytically via HPLC following LAB 1242 methods. The results are summarized in Tables 9a and 9b.

TABLE 9a
Oral Care Compositions (13)-(17)

| Ingredient | (13) | (14) | (15) | (16) | (17) |
|---|---|---|---|---|---|
| Demineralized Water | 53.7 | 54.5 | 54.3 | 54.8 | 54.3 |
| Glycerin | 40 | 40 | 40 | 40 | 40 |
| Xylitol | 3 | 3 | 3 | 3 | 3 |
| Anionic Polymeric Thickening Agent | 0.8 | 0.8 | 0.8 | 0.8 | 0.8 |
| 50% NaOH | 0.5 | 0.5 | 0.5 | 0.5 | 0.5 |
| Benzyl Alcohol | — | — | — | — | — |
| Natural Benzyl Alcohol | 0.5 | — | 0.5 | — | — |
| Zinc Phosphate | 0.187 | — | 0.187 | 0.187 | 0.187 |
| Tetrahydrodiferuloylmethane | 0.3 | 0.15 | 0.15 | 0.15 | 0.15 |
| Sodium Hyaluronate | 0.1 | 0.1 | 0.1 | 0.03 | 0.03 |
| Sodium Methyl Cocoyl Taurate | 0.45 | 0.45 | — | — | — |
| Sodium Ascorbyl Phosphate | 0.1 | 0.1 | 0.1 | 0.1 | 0.1 |
| Cetylpyridinium Chloride(CPC); Initial | 0.075 | 0.075 | 0.015 | 0.075 | 0.075 |
| Castor Oil | — | — | — | — | 0.5 |
| Excipients | Bal. | Bal. | Bal. | Bal. | Bal. |
| Total | 100 | 100 | 100 | 100 | 100 |
| Total Benzyl Alcohol + Taurate | 0.95 | 0.45 | 0.5 | 0 | 0 |
| Ratio Benzyl Alcohol:Taurate | 1:0.9 | — | — | — | — |
| Final CPC | 0.075 | 0.065 | 0.012 | 0.055 | 0.058 |
| Decrease in CPC after Aging (%) | 0 | 13.3 | 20 | 26.7 | 22.7 |

TABLE 9b
Oral Care Compositions (18)-(21)

| Ingredient | (18) | (19) | (20) | (21) |
|---|---|---|---|---|
| Demineralized Water | 54.0 | 54.1 | 54.0 | 54.5 |
| Glycerin | 40.5 | 40 | 40 | 40 |
| Xylitol | 3 | 3 | 3 | 3 |
| Anionic Polymeric Thickening Agent | 0.8 | 0.8 | 0.8 | 0.8 |
| 50% NaOH | 0.5 | 0.5 | 0.5 | 0.5 |
| Benzyl Alcohol | 0.51 | — | 0.5 | — |
| Natural Benzyl Alcohol | — | 0.5 | — | — |
| Zinc Phosphate | 0.1895 | 0.187 | — | — |
| Tetrahydrodiferuloylmethane | 0.15 | 0.3 | 0.15 | 0.15 |
| Sodium Hyaluronate | 0.1 | 0.1 | 0.1 | 0.1 |
| Sodium Methyl Cocoyl Taurate | — | — | 0.45 | 0.45 |
| Sodium Ascorbyl Phosphate | 0.1 | 0.1 | 0.1 | 0.1 |
| Cetylpyridinium Chloride | 0.015 | 0.075 | 0.075 | 0.075 |
| Castor Oil | — | — | — | — |
| Excipients | Bal. | Bal. | Bal. | Bal. |
| Total | 100 | 100 | 100 | 100 |
| Total Benzyl Alcohol + Taurate | 0.51 | 0.5 | 0.95 | 0.45 |
| Ratio Benzyl Alcohol:Taurate | — | — | 1:0.9 | — |
| Final CPC | 0.012 | 0.056 | 0.0705 | 0.0656 |
| Decrease in CPC after Aging (%) | 20 | 25.3 | 6 | 12.5 |

As illustrated in Tables 9a and 9b, it was surprisingly and unexpectedly discovered that the combination of benzyl alcohol and taurate improved the stability of the CPC in the oral care compositions. Particularly, weight ratios of benzyl alcohol to taurate of about 1:1 or 1:0.9 resulted in improved stability of CPC. Without being bound by theory, it is believed that the taurate and benzyl alcohol helps the CPC remain in its active form by increasing the solubility of CPC and preventing the CPC from forming complexes with other ingredients.

The present disclosure has been described with reference to exemplary implementations. Although a limited number of implementations have been shown and described, it will be appreciated by those skilled in the art that changes may be made in these implementations without departing from the principles and spirit of the preceding detailed description. It is intended that the present disclosure be construed as including all such modifications and alterations insofar as they come within the scope of the appended claims or the equivalents thereof

What is claimed is:

1. An oral care composition, comprising:
an orally acceptable vehicle;
hyaluronic acid; and
one or more benefit agents.

2. The oral care composition of claim 1, wherein the hyaluronic acid comprises one or more of hyaluronic acid, a salt of hyaluronic acid, a derivative of hyaluronic acid, or combinations thereof.

3. The oral care composition of claim 1, wherein the hyaluronic acid is present in an amount effective to treat, prevent, or inhibit inflammation of the oral cavity, preferably, the hyaluronic acid is present in an amount of from about 0.05 to about 0.5 wt. %, based on the total weight of the composition.

4. The oral care composition of claim 1, wherein the hyaluronic acid comprises at least low-molecular weight hyaluronic acid.

5. The oral care composition of claim 1, wherein the orally acceptable vehicle comprises a combination of the thickening agent, the humectant, the solvent, the pH modifying agent, and the flavorant.

6. The oral care composition of claim 5, wherein the thickening agent comprises an acrylate copolymer, an acrylate-alkyl acrylate copolymer, or combinations thereof.

7. The oral care composition of claim 5, wherein the thickening agent is present in an amount of from about 0.01 to about 30 wt. %, based on the total weight of the composition.

8. The oral care composition of claim 5, wherein the humectant comprises one or more of xylitol, glycerin, or combinations thereof.

9. The oral care composition of claim 5, wherein the humectant is present in an amount of from about 25 to about 55 weight wt. %, based on the total weight of the composition.

10. The oral care composition of claim 8, wherein the oral care compositions an amount of water, and wherein the oral care composition has a weight ratio of water to glycerin is less than or equal to 1.6:1.

11. The oral care composition of claim 1, wherein the benefit agents comprise one or more of a zinc source, vitamin C, tetrahydrodiferuloylmethane, clove oil, hemp seed oil, one or more antibacterial agents, or combinations thereof.

12. The oral care composition of claim 11, wherein the benefit agent comprises from about 0.01 to about 5 wt. %, based on the total weight of the oral care composition, of the zinc source, the zinc source comprising zinc phosphate hydrate.

13. The oral care composition of claim 11, wherein the benefit agent comprises from about 0.01 to about 5 wt. %, of vitamin C, wherein the vitamin C comprises sodium ascorbyl phosphate.

14. The oral care composition of claim 1, wherein the one or more benefit agents comprises:
clove bud oil, and
benzyl alcohol,
wherein the oral care composition is in the form of a pre-brushing treatment.

15. The oral care composition of claim 14, wherein the benefit agent comprises from about 0.1 to about 2 wt. %, based on the total weight of the oral care composition, of cetylpyridinium chloride.

16. An oral care composition comprising:
an orally acceptable vehicle;
hyaluronic acid;
one or more benefit agents comprising benzyl alcohol; and
a taurate surfactant.

17. The oral care composition of claim 16, wherein the benzyl alcohol and the taurate surfactant and/or a salt thereof are present in a weight ratio of from about 1:0.2 to about 1:2.

18. A method for treating, preventing, or inhibiting dry mouth, inflammation, and/or irritation or discomfort of the oral cavity or a surface thereof, the method comprising contacting the oral cavity or the surface thereof with the oral care composition of claim 16.

19. A method for improving stability of cetylpyridinium chloride in oral care compositions including hyaluronic acid, the method comprising contacting the oral care compositions including the hyaluronic acid with a combination of benzyl alcohol and taurate or a salt thereof, wherein the combination is configured to maintain the cetylpyridinium chloride in an active form.

20. The method of claim 19, wherein the benzyl alcohol and the taurate or a salt thereof are present in a weight ratio of from about 1:0.5; about 1:1.5.
